(19) United States
(12) Patent Application Publication
(10) Pub. No.: US 2005/0244951 A1
(43) Pub. Date: Nov. 3, 2005

(54) Array hybridization chamber with disassembly feature and method

(76) Inventors: Gary B. Gordon, Saratoga, CA (US); Magdalena A. Bynum, San Jose, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/835,650

(22) Filed: Apr. 30, 2004

(51) Int. Cl. C12M 1/34

(52) U.S. Cl. 435/287.2; 435/288.3; 422/102

(57) ABSTRACT

The present invention provides an array hybridization apparatus including a gasket slide and an array slide. The array slide is adapted to be disposed closely adjacent the gasket slide to form a hybridization chamber for retaining an analyte solution in close proximity to an array. At least one of the gasket slide and the array slide has a disassembly feature which aids in disassembling the hybridization chamber. The invention also provides a method for disassembling an array hybridization chamber.

### FIELD OF THE INVENTION

The invention relates generally to microarrays, which are useful in performing biochemical assays, and other applications. More specifically, the invention relates to a hybridization chamber for microarrays which has a feature facilitating disassembly of the hybridization chamber.

### BACKGROUND OF THE INVENTION

Polynucleotide arrays (such as DNA or RNA arrays) are known and are used, for example, as diagnostic or screening tools. Such arrays include regions of usually different sequence polynucleotides arranged in a predetermined configuration on an array slide. These regions (sometimes referenced as “array features”) are positioned at respective locations (“addresses”) on the array slide. In use, the arrays, when exposed to a sample, will exhibit an observed binding or hybridization pattern. This binding pattern can be detected upon interrogating the array. For example, all polynucleotide targets (for example, DNA) in the sample can be labeled with a suitable label (such as a fluorescent dye), and the fluorescence pattern on the array accurately observed following exposure to the sample. Assuming that the different sequence polynucleotides were correctly deposited in accordance with the predetermined configuration, the observed binding pattern will be indicative of the presence and/or concentration of one or more polynucleotide components of the sample.

Biopolymer arrays can be fabricated by depositing previously obtained biopolymers (such as from synthesis or natural sources) onto an array slide, or by in situ synthesis methods. Methods of depositing obtained biopolymers include dispensing droplets to an array slide from dispensers such as pins or capillaries (such as described in U.S. Pat. No. 5,807,522), thermal injets, or pulse jets (such as a piezoelectric inkjet head, as described in PCT publications WO 95/25116 and WO 98/41531, and elsewhere). For in situ fabrication methods, multiple different reagent droplets are deposited stepwise from drop dispensers at a given target location in order to form the final feature (hence a probe of the feature is synthesized on the array substrate). The in situ fabrication methods include those described in U.S. Pat. No. 5,449,754 for synthesizing peptide arrays, and described in WO 98/41531 and the references cited therein for polynucleotides. The in situ method for fabricating a polynucleotide array typically follows, at each of the multiple different addresses at which features are to be formed, the same conventional iterative sequence used in forming polynucleotides from nucleoside reagents on a support by means of known chemistry. This iterative sequence is as follows: (a) coupling a selected nucleoside through a phosphite linkage to a functionalized support in the first iteration, or a nucleoside bound to the array slide (i.e. the nucleoside-modified array slide) in subsequent iterations; (b) optionally, blocking unreacted hydroxyl groups on the array slide bound nucleoside; (c) oxidizing the phosphite linkage of step (a) to form a phosphate linkage; and (d) removing the protecting group (“deprotection”) from the now array slide bound nucleoside coupled in step (a), to generate a reactive site for the next cycle of these steps. The functionalized support (in the first cycle) or deprotected coupled nucleoside (in subsequent cycles) provides an array slide bound moiety with a linking group for forming the phosphite linkage with a next nucleoside to be coupled in step (a). Final deprotection of nucleoside bases can be accomplished using alkaline conditions such as ammonium hydroxide, in a known manner.

The foregoing chemistry of the synthesis of polynucleotides is described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. No. 4,458,066, U.S. Pat. No. 4,500,707, U.S. Pat. No. 5,153,319, U.S. Pat. No. 5,869,643, EP 0294196, and elsewhere. In both cases, the arrays can be generated in a way that multiple arrays coexist on one slide.

Array slides are typically employed for deposition and in situ arrays. They generally comprise a separate slide with attached or fixed arrays. However, in some cases, the arrays may be deposited and/or attached onto the same slide as the gasket. In other cases a separate gasket slide may be employed.

Gasket slides used for arrays are important because they enclose the analyte solutions used for the binding reactions. A variety of slide materials have been proposed. For instance, the standard slide may comprise a glass slide or similar type material. A typical gasket and/or spacer is then disposed onto the glass, formed onto the glass, adhered to the glass, or may be pre-cut and attached to the glass. These gasket slides are designed to provide spacing so that the analyte solutions reside in a region defined as a hybridization chamber. In the case of a protein array the hybridization chamber is typically referred to as the binding chamber; but for the purposes of the present description, “hybridization chamber” will be used to refer to the chamber formed by the combination of the gasket slide and the array slide, whether the array is a protein array, a polynucleotide array, or other type of molecular array.

The gasket slide and the array slide are most often separated by inserting a wedge between the gasket slide and the array slide. The wedge is then twisted and the gasket slide is separated from the array slide. This technique is problematic since it requires care and manual dexterity so as not to damage the array or lose the solutions held within the gaskets or chambers. Therefore, there is a substantial need to provide an improved hybridization chamber and method for separation of array slides from gasket slides.

It, therefore, would be desirable to facilitate assembly and disassembly of the hybridization chamber and provide a method that meets the above described needs. These and other problems are addressed by the present invention.

### SUMMARY OF THE INVENTION

The invention addresses the aforementioned deficiencies in the art, and provides novel features for hybridization chambers that aid in disassembly of the hybridization chambers. The present invention provides an array hybridization apparatus including a gasket slide and an array slide. The array slide is adapted to be disposed closely adjacent the gasket slide to form a fluid-tight chamber (the hybridization chamber) for retaining an analyte solution in close proximity to an array disposed on a surface of the array slide. At least one of the gasket slide and the array slide has a disassembly feature which aids in disassembling the hybridization chamber. The disassembly feature is a structural element such as, e.g., a bevel, slot, groove, facet, notch, cutout, or the like, at or adjacent to an edge of at least one of the gasket slide and the array slide. The disassembly feature provides for a gap along an edge of the assembled hybridization chamber. The gap may be any cavity, cleft, indentation, hollow, recess, or other opening that is configured to accept entry of a disassembly means to facilitate separation of the gasket slide and array slide.

The invention also provides a method for disassembling an array hybridization chamber formed by a gasket slide disposed closely adjacent an array slide, wherein at least one of the array slide and gasket slide has a disassembly feature. The method comprises inserting a disassembly means into the disassembly feature and providing a force to the disassembly means to separate the array slide from the gasket slide. Disassembly means may be fingernail, a spatula, pick, a lever, a tweezers, a screwdriver, or other thin tool, or the like that is shaped to allow it to fit into the disassembly feature to apply a force to urge the gasket slide and the array slide apart. The disassembly may be performed in a bath (under a liquid, e.g. solvent, buffer, or wash liquid) or dry (e.g. in the air). Typically, the disassembly is followed by rinsing and drying of the array and interrogating the array.

Additional objects, advantages, and novel features of this invention shall be set forth in part in the descriptions and examples that follow and in part will become apparent to those skilled in the art upon examination of the following specifications or may be learned by the practice of the invention. The objects and advantages of the invention may be realized and attained by means of the instruments, combinations, compositions and methods described herein and/or particularly pointed out in the appended claims.

### BRIEF DESCRIPTION OF THE DRAWINGS

These and other features of the invention will be understood from the description of
representative embodiments of the method herein and the disclosure of illustrative apparatus
for carrying out the method, taken together with the Figures, wherein

FIG. 1 illustrates a slide carrying an array, such as may be used in the present invention;

FIG. 2 is an enlarged view of a portion of FIG. 1 showing ideal spots or features;

FIG. 3 shows features on the surface of a slide;

FIG. 4 illustrates an array hybridization apparatus having facets as a disassembly feature;

FIG. 5 depicts an embodiment of the invention in which a gasket slide has a disassembly feature; and

FIG. 6 shows a gap provided for by disassembly features of an array hybridization apparatus.

To facilitate understanding, identical reference numerals have been used, where practical, to designate corresponding elements that are common to the Figures. Figure components are not drawn to scale.

### DETAILED DESCRIPTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible in the present invention that steps may be executed in different sequence where this is logically possible. However, the sequence described below is typical.

It must be noted that, as used in the specification and the appended claims, the singular forms “a,” “an” and “the” include plural referents unless the context clearly dictates otherwise. Thus, for example, reference to “an array” includes a plurality of arrays. In this specification and in the claims that follow, reference will be made to a number of terms that shall be defined to have the following meanings unless a contrary intention is apparent.

A “biopolymer” is a polymer of one or more types of repeating units. Biopolymers are typically found in biological systems (although they may be made synthetically) and particularly include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and UNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups). A “peptide” is used to refer to an amino acid multimer of any length (for example, more than 10, 10 to 100, or more amino acid units). A biomonomer fluid or biopolymer fluid reference a liquid containing either a biomonomer or biopolymer, respectively (typically in solution).

A “set” or “sub-set” of any item (for example, a set of features) may contain one or more than one of the item (for example, a set of features may contain one or more such features). An “array”, unless a contrary intention appears, includes any one, two or three dimensional arrangements of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the array slide at the various regions. However, either of the “target” or “target probes” may be the one that is to be evaluated by the other (thus, either one could be an unknown mixture of polynucleotides to be evaluated by binding with the other). An “array layout” refers collectively to one or more characteristics of the features, such as feature positioning, one or more feature dimensions, and some indication of a moiety at a given location. “Hybridizing” and “binding”, with respect to polynucleotides or polypeptides, are used interchangeably.

The term “adjacent” or “adjacent to” refers to a component or element that is near, next to or adjoining. For instance, a gasket may be adjacent to a spacer.

The term “substantially deformable”, “compressible” or “deformable” shall all have a similar meaning.

The term “slide” refers to any number of materials having at least one planar surface capable of contacting a gasket or spacer. The term shall be broad based to include array slides, polymeric materials, silica based materials, plastics etc.. It's important that the “slide” maintain a certain amount of rigidity to compress or deform the gasket and contact the spacer. In certain instances a “slide” will be transparent to allow light to pass through its medium. However, this is not required. The surface may also contain a reflective coating. Also, the “slide” must be capable in certain instances to allow for the mounting or construction of an array or gasket on its surface. Although in certain cases this will not be required if the array is constructed on a separate surface.

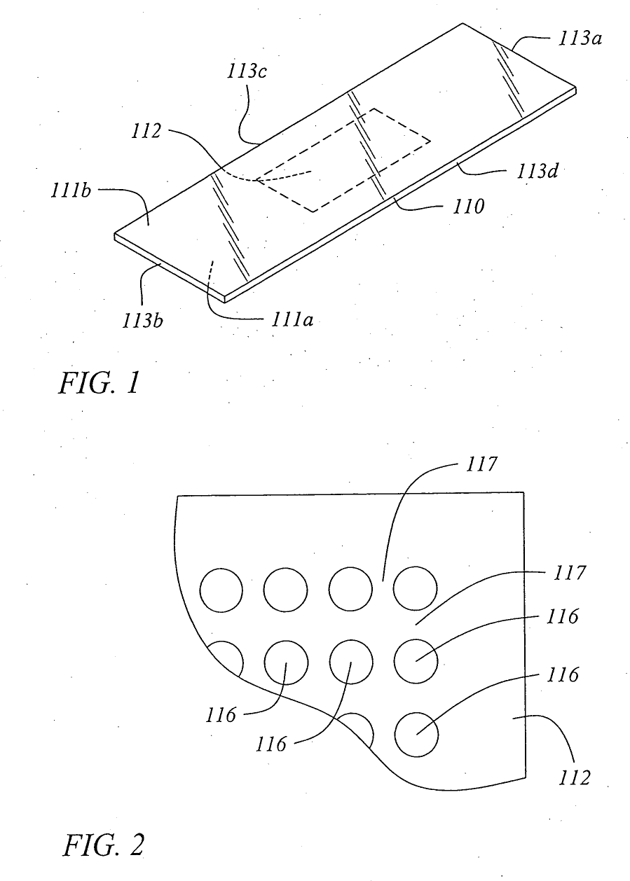

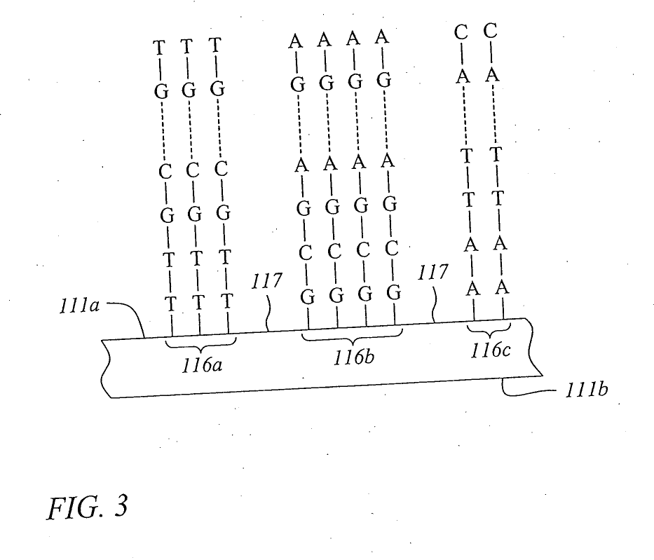

Referring first to FIGS. 1, 2, and 3, typically the methods and apparatus of the present invention generate or use an array slide 110 carrying an array 112 disposed on a rear surface 111a of an array slide 110. It will be appreciated though, that more than one array (any of which are the same or different) may be present on the rear surface 111a, with or without spacing between such arrays. Note that one or more of the arrays 112 together will cover the entire region of the rear surface 111a, with regions of the rear surface 111a adjacent to the opposed sides 113c, 113d and the leading end 113a and the trailing end 113b of the slide 110. A front surface 111b of the array slide 110 does not carry any of the arrays 112. Each of the arrays 112 can be designed for testing against any type of sample, whether a trial sample, reference sample, a combination of them, or a known mixture of polynucleotides (in which latter case the arrays may be composed of features carrying unknown analytes or sequences to be evaluated). The array slide 110 may be of any shape, and any holder used with it adapted accordingly, although the array slide 110 will typically be rectangular in practice.

The array 112 contains multiple spots or features 116 of biopolymers in the form of small molecules such as organic drugs, polynucleotides, polypeptides or proteins. A typical array may contain from more than ten, more than one hundred, more than one thousand, or more than ten thousand features, or even more than one hundred thousand features. All of the features 116 may be different, or some or all could be the same. Features may comprise oligonucleotides and/or proteins/peptides or other biopolymers known in the art. In the case where the array 112 is formed by the conventional in situ or deposition of previously obtained moieties, as described above, by depositing for each feature at least one droplet of reagent such as by using a pulse jet such as an inkjet type head, interfeature areas 117 will typically be present which do not carry any polynucleotide. It will be appreciated though, that the interfeature areas 117 could be of various sizes and configurations. Each feature carries a predetermined polynucleotide (which includes the possibility of mixtures of polynucleotides). As per usual, A, C, G, T represent the usual nucleotides. It will be understood that there may be a linker molecule (not shown) of any known types between the rear surface 111a and the first nucleotide.

The array slide 110 may also carry on the front surface 111b or on the rear side 111a, an identification code such as a bar code (not shown) printed on an array slide. The bar code contains an identification of the array 112 and either contains or is associated with, array layout or layout error information which may be referenced using the identification code.

For the purpose of the discussions below, it will be assumed (unless the contrary is indicated) that the array 112 is a polynucleotide or protein array formed by the deposition of previously obtained polynucleotides or proteins using pulse jet deposition units. However, it will be appreciated that an array of other polymers or chemical moieties generally, whether formed by multiple cycles in situ methods adding one or more monomers per cycle, or deposition of previously obtained moieties, or by other methods, may be present instead.

Referring now to FIG. 4, an array slide 110 (such as depicted in FIG. 1) is shown above a gasket slide 120. A gasket 124, typically made of a conformable material, is disposed on a surface of the gasket slide 120. The gasket slide 120 also has a structural element in the form of facets 122 at the four corners of the gasket slide 120. The facets are a disassembly feature in accordance with the present invention.

In use, a small amount of analyte solution or other solution is placed oh the gasket slide 120 within an area of the slide surface defined by the gasket 124. The gasket slide 120 (with the analyte solution) is then carefully covered with the array slide 110, held together by clamps or other means well known in the art, thus assembling the hybridization chamber. The gasket slide/array slide assembly is then subjected to appropriate conditions for a binding reaction or other reaction to occur. When the reaction has proceeded to an desired stopping point, the assembly is disassembled in accordance with the methods described herein.

FIG. 5 illustrates another embodiment of the invention, in which an array slide 110 is positioned above a gasket slide 120 prepared to receive the array slide 110. In the pictured embodiment, the beveled edge 126 is a structural feature present as part of the array slide 110 and of the gasket slide 120. Thus, both the array slide and the gasket slide have a disassembly feature in the embodiment of FIG. 5.

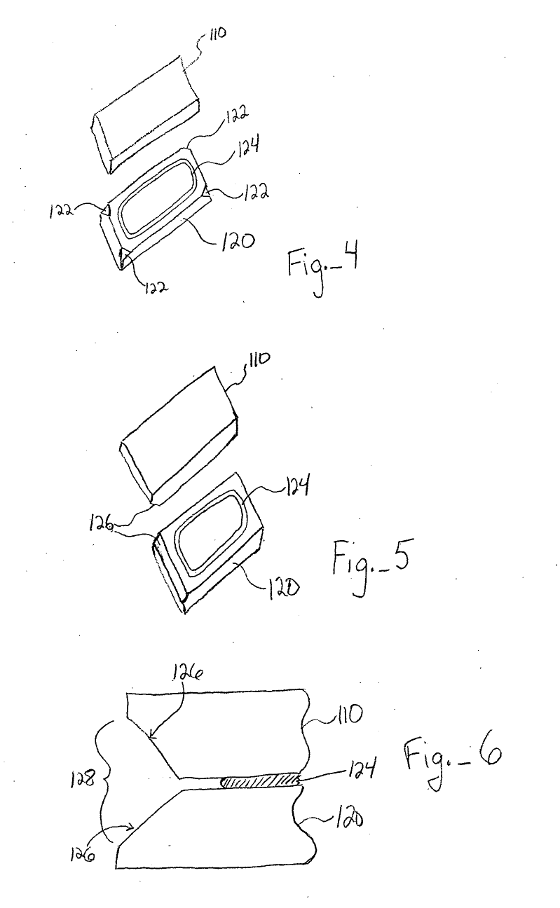

FIG. 6 shows a portion of a side view of an embodiment like that shown in FIG. 5. In FIG. 6, the array slide 110 is in place on the gasket slide 120 to form a tight seal against the gasket 124, thus forming the assembled hybridization chamber. The bevel 126 on both the array slide 110 and the gasket slide 120 provides a gap 128 at the edge of the assembly. The side view close up view of FIG. 6 emphasizes (in a very conceptual way, since the figures are not to scale) the additional clearance of the gap 128 versus the thickness of the gasket 124. The disassembly feature may be in the nature of a space, an opening, a cutout, a slot, a groove, a niche, a facet, a bevel, a crevice, indentation, hollow, furrow, trench, fissure, cleft, or any other such feature that provides a gap when the hybridization chamber is assembled. The resulting gap should be configured to provide a way to apply a force to separate the gasket slide from the array slide.

After having described the apparatus of the invention, a description of the method of the invention is now in order. The current invention provides methods for disassembling an array hybridization chamber formed by a gasket slide closely adjacent to an array slide, wherein at least one of the array slide and gasket slide has a disassembly feature, such as is shown in FIG. 6. The method comprises inserting a disassembly means into the disassembly feature (gap 128) and providing a force to separate the array slide from the gasket slide. The disassembly means may be fingernail, a spatula, a wedge, a pick, a lever, a tweezers, a screwdriver, or other thin tool, or the like that is shaped to allow it to fit into the disassembly feature to apply a force to urge the gasket slide and the array slide apart. In certain embodiments, the disassembly means is any tool that is shaped to allow it to fit into the gap to apply a force to disassemble the array hybridization chamber. In some embodiments, the disassembly of the array hybridization chamber may be done with the array chamber disposed in a bath (e.g. to keep the surface wet or to quickly rinse off analyte solution while reducing risk of contaminating other portions of the array slide). In other embodiments, the array hybridization chamber may be disposed in any suitable location, e.g. in the air, not submerged in a bath. The disassembly of the array hybridization chamber is typically followed by further steps, including rinsing, drying, and interrogation of the array.

In previous array systems employing array slides and mating gasket slides, it was observed that after the binding reaction takes place, it was frequently difficult to disassemble the reaction chambers, because the slides had become bonded together by hydrogen bonding, van der waals forces, ionic bonding, by vacuum, or otherwise stuck together.

The present invention addresses this problem and provides novel features for hybridization chambers that aid in disassembly of the hybridization chambers. The present invention provides a gasket slide and an array slide, wherein the array slide is adapted to be disposed closely adjacent to the gasket slide (e.g. with the gasket interposed between the gasket slide and the array slide) to form a fluid-tight chamber (the hybridization chamber) for retaining an analyte solution in close proximity to an array (e.g. in contact with the array) disposed on a surface of the array slide. At least one of the gasket slide and the array slide has a disassembly feature which aids in disassembling the hybridization chamber.

The disassembly feature is a structural element such as, e.g., a bevel, slot, groove, facet, notch, cutout, or the like, at or adjacent to an edge of at least one of the gasket slide and the array slide. The disassembly feature may be present at just one portion of one edge of a slide, or may be located at a plurality of sides at or near the edge of a slide. In certain embodiments, the gasket is very thin (e.g. in the range about 40 microns to about 400 microns, typically in the range 50 microns to about 250 microns, more typically in the range from about 50 microns to about 150 microns). In other embodiments, the gasket may be in the range of about 0.5 mm to about 2.5 mm thick, more typically in the range from about 0.6 mm to about 1.5 mm thick.

The examples described herein are put forth so as to provide those of ordinary skill in the art with a complete disclosure and description of how to perform the methods and use the compositions disclosed and claimed herein. Efforts have been made to ensure accuracy with respect to numbers (e.g., amounts, temperature, etc.) but some errors and deviations should be accounted for. The practice of the present invention will employ, unless otherwise indicated, conventional techniques of synthetic organic chemistry, biochemistry, molecular biology, and the like, which are within the skill of the art. Such techniques are explained fully in the literature.

While the foregoing embodiments of the invention have been set forth in considerable detail for the purpose of making a complete disclosure of the invention, it will be apparent to those of skill in the art that numerous changes may be made in such details without departing from the spirit and the principles of the invention. Accordingly, the invention should be limited only by the following claims.

All patents, patent applications, and publications mentioned herein are hereby incorporated by reference in their entireties.

1. An array hybridization apparatus comprising
a gasket slide and an array slide, wherein the array slide is adapted to be disposed closely adjacent the gasket slide to form a hybridization chamber, wherein at least one of the gasket slide and the array slide has a disassembly feature which aids in disassembling the hybridization chamber.

2. The array hybridization apparatus according to claim 1, wherein the disassembly feature is a structural element selected from the group consisting of a bevel, a slot, a groove, a facet, a notch, and a cutout.

3. The array hybridization apparatus according to claim 1, wherein the disassembly feature provides for a gap configured to accept a disassembly means.

4. The array hybridization apparatus according to claim 3, wherein the disassembly means is selected from the group consisting of a fingernail, a spatula, a wedge, a pick, a lever, a tweezers, and a screwdriver.

5. The array hybridization apparatus according to claim 1, wherein, when the array slide is disposed closely adjacent the gasket slide to form the hybridization chamber, the disassembly feature forms a gap configured to accept a disassembly means.

6. The array hybridization apparatus according to claim 5, wherein the disassembly means is selected from the group consisting of a fingernail, a spatula, a wedge, a pick, a lever, a tweezers, and a screwdriver.

7. The array hybridization apparatus according to claim 1, wherein the disassembly feature is at or adjacent to an edge of at least one of the gasket slide and the array slide.

8. The array hybridization apparatus according to claim 1, wherein the gasket slide comprises a gasket having a thickness in the range of about 40 microns to about 400 microns.

9. The array hybridization apparatus according to claim 1, wherein the array slide comprises a peptide array.

10. The array hybridization apparatus according to claim 1, wherein the array slide comprises a polynucleotide array.

11. A method for disassembling an array hybridization chamber, the method comprising:
a) inserting a disassembly means into a gap provided by a disassembly feature, and
b) providing a force to the disassembly means to disassemble the array hybridization chamber.

12. A method according to claim 11, wherein the disassembly means is selected from the group consisting of a fingernail, a spatula, a pick, a lever a tweezers, a screwdriver, and a wedge.

13. A method according to claim 11, wherein the disassembly means is a tool that is shaped to allow it to fit into the gap to apply a force to disassemble the array hybridization chamber.

14. A method according to claim 11, wherein the array hybridization chamber comprises an array slide disposed adjacent a gasket slide, and the step of providing the force results in the gasket slide and the array slide being separated.

15. A method according to claim 11, wherein the disassembly feature is a structural element selected from the group consisting of a bevel, a slot, a groove, a facet, a notch, and a cutout.

16. A method according to claim 11, wherein the array hybridization chamber is disposed in a bath when the force is provided.
